(19) United States
(12) Patent Application Publication
(10) Pub. No.: US 2011/0077601 A1
(43) Pub. Date: Mar. 31, 2011

(54) Quadraglide Syringe

(76) Inventor: Andrew Jobson, Melissa, TX (US)

(21) Appl. No.: 12/571,045

(22) Filed: Sep. 30, 2009

(51) Int. Cl. A61M 5/315

(52) U.S. Cl. 604/222

(57) ABSTRACT

A syringe assembly may include a syringe barrel, a plunger rod to cooperate with the syringe barrel, a deformable seal member having a deformable depression and a lobe member to cooperate with the syringe barrel. The lobe member may be a corner lobe member, and the syringe assembly may be a cone shaped tip to cooperate with the seal member. The cone shaped tip may cooperate with the depression, and the depression may be a distal depression.

### FIELD OF THE INVENTION

The present invention relates to a syringe assembly or other medical device wherein it is desirable to move a fluid through a conduit.

### BACKGROUND

A conventional syringe consists of a cylindrical barrel member, most commonly made of a thermoplastic material such as polypropylene. The conventional syringe typically has a reduced diameter distal end adapted to receive a needle thereon and an open proximal end adapted to receive a plunger rod assembly therein. The plunger rod assembly generally consists of an elongate plunger rod which is commonly made of a thermoplastic material such as polypropylene and a flexible plunger tip on the distal end thereof. The plunger tip is commonly made of an elastomeric material such as butyl or ethylene rubber.

One of the functions of the plunger tip is to provide a substantially air tight seal with the interior surface of the syringe barrel so that movement of the plunger tip distally and proximally in the syringe barrel will cause medication, blood or other fluids to be drawn into or forced out of the distal end of the syringe barrel. The plunger tip is moved through the inside of the syringe barrel by applying axial force to the rigid plunger rod which has sufficient length to be accessible through the proximal end of the syringe barrel. Therefore, the plunger tip is designed to have sufficient flexibility so that it will create a seal with the interior surface of the syringe barrel without creating excessive resistance to the movement of the plunger tip through the syringe barrel.

In order to ensure an air tight seal with the interior surface of the syringe barrel, plunger tips are typically manufactured with an outside diameter which is significantly larger than the inside diameter of the syringe barrel. The plunger tip is designed such that when the plunger tip is introduced into the syringe barrel, the plunger tip is compressed sufficiently to provide adequate pressure between the barrel and the plunger tip to seal this interface. As a result of the plunger tip design, the interface of the plunger tip and the barrel maintains, at all times, a sealing pressure sufficient to withstand the challenges of filling and injecting even though the same magnitude of sealing pressure may not be required when the syringe is not in use. A major drawback to this approach is that the increased diameter of the plunger tip increases the frictional resistance to movement of the plunger tip in the barrel.

This invention is a Class II Medical Device being a disposable, single use plastic syringe. This is a class D24/130 classification patent application, being “Medical and Laboratory Equipment”, sub level 130 “Syringe, catheter or needle component”. This design was established through input from clinicians, doctors, anesthetists and experts in the syringe manufacturing industry.

Design requirements for the industry that fills syringes (potential customer) and then sells a syringe that is filled, sterilized and ready for use were established as follows:

(i) A standard butyl rubber plunger tip tends to expose high Cyto-toxicity issues that may affect the sterilization integrity. The syringe design needs to incorporate a plunger material and design methods to lower the Cyto-toxicity risk.
(ii) The traditional Butyl rubber tip has extractable and leachable chemicals that can affect the PH value of the fill. The material selection of the seal as well as the exposure of this material to the fill solution should be minimized.
(iii) A standard rubber tip seals by compressing two or more raised radii, the seal relies totally on this compression and the subsequent forces exerted by the plunger on the barrel wall are considerable and sometimes cause a bulge to emerge when a syringe is steam sterilized as is the case in the prefilled syringe manufacturing process. The syringe design needs to minimize or alleviate the bulging of the syringe barrel.

A design that lowers the manufacturing cost and subsequent price for bulk syringe sub-assemblies is needed. Currently, the independent syringe prefill companies (companies that do not manufacture the syringe itself) are seeking lower costs for the syringes they fill.

Traditional plunger tips are designed with two or more raised, radiused ribs that seal off against the syringe barrel bore because of an interference fit. Traditional plunger tips are made from Butyl rubber which will not slide on plastic so the rubber tip is coated with a liquid silicone before it is assembled into a syringe. This lubrication by itself is still insufficient and the inside of the syringe barrel must also be sprayed with liquid silicone to achieve a smooth action of the plunger.

The purpose of more than one raised rib, is so that there is a back-up seal in the event the first seal fails or leaks. A traditional plunger tip is a single piece that is shaped to evacuate as much of the fluid inside the syringe barrel as possible when the plunger is fully depressed.

Traditional syringes are lubricated with liquid silicone and the glide force is dependent on how consistently the syringe barrel was coated with the liquid silicone.

### SUMMARY

A syringe assembly may include a syringe barrel, a plunger rod to cooperate with the syringe barrel, a deformable seal member having a deformable depression and a lobe member to cooperate with the syringe barrel.

The lobe member may be a corner lobe member, and the syringe assembly may be a cone shaped tip to cooperate with the seal member.

The cone shaped tip may cooperate with the depression, and the depression may be a distal depression.

### BRIEF DESCRIPTION OF THE DRAWINGS

The invention may be understood by reference to the following description taken in conjunction with the accompanying drawings, in which, like reference numerals identify like elements, and in which:

FIG. 1a illustrates an exploded view of the syringe assembly of the present invention;

FIG. 1b illustrates a cross-sectional view of the syringe assembly of the present invention;

FIG. 2 illustrates a cross-sectional view of the seal member of the present invention in a first position;

FIG. 3 illustrates a cross-sectional view of the seal member of the present invention in a second position;

FIG. 4 illustrates a distal end view of the syringe assembly of the present invention.

FIG. 5 illustrates a cross-sectional view of the seal member and the syringe assembly of the present invention.

### DETAILED DESCRIPTION

The design requirements for the present invention are for a syringe that has lower and more consistent performance in the area of plunger “break force” (the force needed to start the plunger movement down the syringe) and “glide force” (the force needed after the initial start of the plunger movement throughout the remainder of the injection). The design of the Quadraglide syringe (syringe assembly) addresses these design requirements, and may use mechanical and hydraulic design principles to develop a syringe of superior performance quality, while designing product that has lower manufacturing costs and price to the customer. The plunger seal design addresses the issues of reducing the “break force” and the “glide force” of the Quadraglide syringe. The plunger seal includes four lobes that seal off, two lobes may be positioned against the inner barrel bore, and two lobes may be positioned against the seal tip. The seal is designed to exert less force on the barrel wall than a traditionally designed plunger tip and hydraulically designed so that the pressure the seal exerts on the barrel wall increases as the pressure is increased on the plunger and the subsequent pressure of the fluid inside the syringe increases. This feature is achieved through deepening the radius of the proximal and distal recesses of the seal in relation to the plunger thumb pad, and so increasing the surface area exposed to the fluid pressure. The pressure increase forces the seal lobe against both the barrel wall and the seal tip, so increasing the efficiency of the seal. This feature improves the syringe whenever it is used for a prefilled device and the syringe is steam sterilized. With a traditional plunger design the force the plunger tip exerts against the barrel wall can cause a bulging of the barrel wall during the steam sterilization process as the plastic syringe barrel is annealed. The design of the four lobe seal allows the sealing lobe to collapse easier, resulting on less force exerted against the barrel wall. Because the seal may not be welded or adhered in any way to the seal tip, the seal is allowed to perform naturally and most effectively.

The seal material selection for “normal use” which may be defined as “non protein based fills”, may be silicone USP6 grade 50 shore “A” durometer LIM 6050 or other appropriate material. Since silicone may not stick to plastic and has natural lubricity, the break force and glide forces are lowered and the consistency of the glide force may be enhanced. The Quadraglide syringe may be also lubricated with liquid silicone but does not rely totally on this for repeatable glide force. Since silicone may be a common ingredient of a syringe, and silicone also has very low Cyto-toxicity traits this material seal is ideal for this application. Silicone also has far fewer extractable and leachable chemicals that could potentially affect the PH value of the liquid fill of the syringe. Silicone may not however ideal for every application and may not be used if the fill of the syringe contains proteins, since these may react with the silicone. For these purposes and for syringes that will be used for insulin a Thermo Plastic Elastomeric material may be used.

The seal tip design may be used in two areas, primarily for the reduction of material needed for the seal (and so a reduction in device costs) and secondly the reduction of the seal material surface area to the syringe fill and so reducing the potential cyto-toxicity exposure of the fill as well as minimizing the leachable exposure. The seal tip may be made from the same USP6 Medical Grade Polypropylene as the syringe barrel and plunger. The tip is designed to hold the seal and fill the seal face when the seal is collapsed in order to eliminate areas where air bubbles may be trapped. This reduction of exposure to the area of most risk of cyto-toxicity and leachables is advantageous when the syringe contains fluids for extended periods of times such as prefilled flush syringes. This design factor, along with the seal material selection reduces the effects to the PH value of the fill as a syringe is processed and sterilized in a prefill manufacturing operation. The Quadraglide plunger seal may have four lobes two on the external diameter and two on the inside diameter which equates to a traditional two rib plunger as far as sealing to the barrel. The two lobes on the internal diameter may seal off on the seal tip component, completing the seal between the fluid side of the plunger and the plunger rod.

There are depressions in all four sides of the seal as shown in FIG. 3. However, the depressions are greater/deeper on the side that pushes against the fluid. The reason for this feature is twofold, primarily the depression allows the lobe to collapse easier reducing the force exerted on the barrel wall. However, when the seal pushes against fluid and the pressure in front of the seal increases, the same pressure is exposed to the radius of the depression and forces the sealing lobes harder against the wall of the syringe barrel and the seal tip, thus increasing the seals ability to maintain the fluid in front of the plunger assembly. The advantage of a seal that is a separate component and not adhered to another component is that it will collapse naturally and evenly when used.

All components of the syringe may be injection molded; in the case of the silicone seal, a liquid injection molding process may be used. The seals may be then coated in liquid silicone to a predetermined amount, then fed to automation machinery where they are assembled on the seal tip component. This seal tip assembly is now “snapped” onto the end of the plunger rod which has a feature that will secure the seal tip assembly to the rod. The syringe barrel may be first cleaned by blowing compressed air through a jet that is lowered into the barrel bore, and a vacuum above the blow out jet collects any debris from the cleaning. Once cleaned the inside of the barrel may be then sprayed with liquid silicone and finally the plunger assembly is inserted into the barrel and pushed closed. Once the syringe assembly is completed the syringes are counted and packed in bulk cases or individually packaged in blister packs and cartons for sterilization.

The Quadraglide silicone seal may require less force to move the plunger down the barrel (glide force) than comparable sized general use disposable syringes.

The Quadraglide silicone seal may require less force to start the plunger moving (break force) than comparable sized general use disposable syringes.

Manufacturing costs may be reduced due to minimizing the quantity of the higher priced raw materials.

The materials selected for the seal may have lower leachable quality that may potentially affect the PH value of the fill when the syringe is steam sterilized.

The materials may be selected for the seal have less Cyto-toxicity risks that Butyl rubber.

In the preferred embodiments described herein, the present invention is intended to be used with a conventional syringe. Accordingly, the terms “syringe,” “plunger rod assembly” and “plunger tip” are intended to be construed broadly to include a device having the characteristics or features described above.

Now describing the invention, the “distal end” or “distal portion” of a part or member may be refer to the end or portion which is positioned closest to the patient during use of the device. The terms “proximal” and or “proximal portion” of a part or member is intended to refer to the end or portion of a part or member which is positioned furthest from the patient during use of the device.

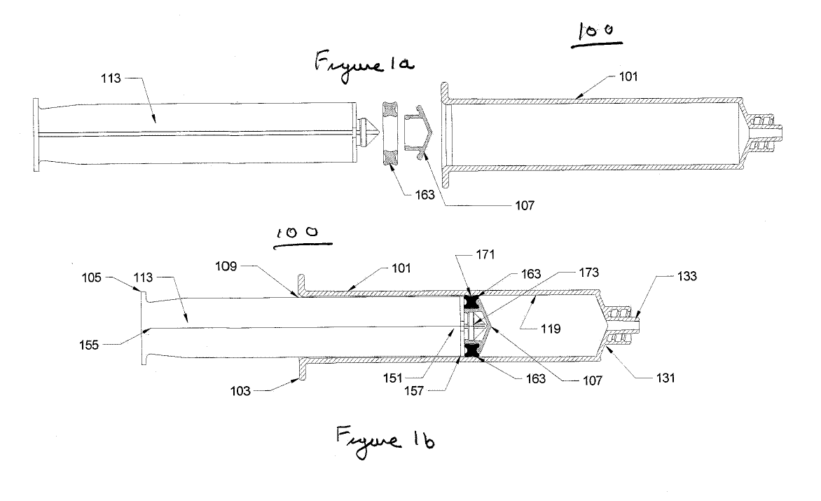

As shown in the drawings, the present invention preferably forms part of a syringe assembly 100. The syringe assembly 100 preferably includes an elongate and tubular syringe barrel 101, and plunger rod assembly 113 received therein. As shown in FIG. 1a,b, the syringe barrel 101 is an elongate and tubular member preferably made of a polypropylene or similar material. The proximal end 109 of the syringe barrel 101 is preferably open to the interior surface 119 of the syringe barrel 101 and includes an enlarged finger flange 103 extending radially outwardly therefrom. The distal end 131 of the syringe barrel 101 has a reduced diameter opening 133 as compared to the interior surface 119 of the syringe barrel 101 and the opening at the proximal end 109 of the syringe barrel 101.

The needle assembly (not shown) is preferably removably mounted on the distal end 131 of the syringe barrel 101. The needle assembly preferably includes a cannula having a sharpened distal needle point thereon and a hub member on the proximal end thereof. The hub member is designed to engage the luer tip and luer skirt on the distal end 131 of the syringe barrel 101 when the needle assembly is mounted thereon.

As shown in the drawings, the plunger rod assembly 151 may include an elongate plunger rod 113 and a flexible plunger tip 107. The plunger rod 113 includes an elongate shaft portion 155 defining a first longitudinal axis 157. The proximal end of the plunger rod 151 includes an enlarged disc-shaped member which forms a thumb rest 105 thereon. An enlarged flange 157 is preferably located at the distal end of the shaft portion 155 of the plunger rod 113. This enlarged flange 157 may be a flat surface in a plane substantially perpendicular to the longitudinal axis 158 of the plunger rod 113. The diameter of this enlarged flange 157 is preferably slightly less than the diameter of the interior surface 119 of the syringe barrel and significantly less than the diameter of the thumb rest 105. The distal portion 159 of the plunger rod 113 extends distally from the shaft portion 155 and enlarged flange 157 of the plunger rod 113.

The distal portion may include a cone shaped tip 107 which may include a circumferentially inclined face which may extend from the distal depression 171 of the seal member 163. A central axial frame member 173 extends from the apex of the cone shaped tip 107 and may extend to the distal side of the enlarged flange 157, and a radial frame member 175 may extend radially to support the opposing sides of the seal member 163.

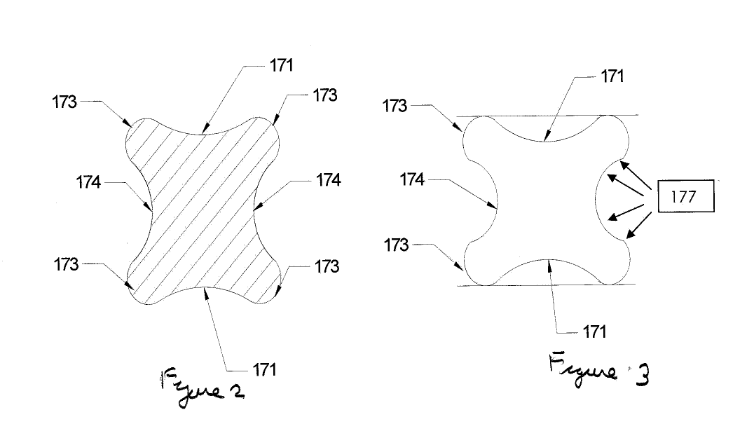

FIG. 2 illustrates a flexible and deformable seal member 163 which may include a central section 175 which may include corner lobes 173 which may extend radially from the center section 175. The corner lobes 173 may define a depression 171 which may be deformed based upon the applied pressure resulting from the pressure force 177 generated by the movement of the plunger rod 113 against the fluid within the syringe barrel 101. As a result of this pressure force 177, the distal depression 171 may be deeper or more deformed than the proximal depression 171 and may outer and inner depressions 171.

FIG. 3 illustrates a force 177 being applied to the distal depression 171 and the resulting compression of the distal depression 171 and illustrates a flexible and deformable seal member 163 which may include a central section 175 which may include corner lobes 173 which may extend radially from the center section 175. The corner lobes 173 may define a depression 171 which may be deformed based upon the applied pressure resulting from the pressure force 177 generated by the movement of the plunger rod 113 against the fluid within the syringe barrel 101. As a result, the distal depression 171 may be deeper or more deformed than the proximal depression 171 and may outer and inner depressions 171.

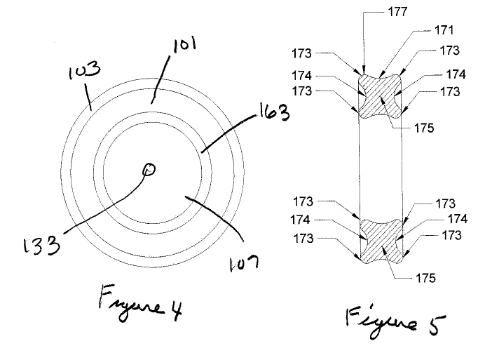

FIG. 4 illustrates a distal end view of the syringe 100 of the present invention.

FIG. 5 illustrates a flexible and deformable seal member 163 which may include a central section 175 which may include corner lobes 173 which may extend radially from the center section 175. The corner lobes 173 may define a depression 171 which may be deformed based upon the applied pressure resulting from the pressure force 177 generated by the movement of the plunger rod 113 against the fluid within the syringe barrel 101. As a result of this pressure force 177, the distal depression 171 may be deeper or more deformed than the proximal depression 171 and may outer and inner depressions 171.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed.

1) A syringe assembly, comprising:
a syringe barrel;
a plunger rod to cooperate with the syringe barrel;
a deformable seal member having a deformable depression and a lobe member to cooperate with the syringe barrel.

2) A syringe assembly as in claim 1, wherein the lobe member is a corner lobe member.

3) A syringe assembly as in claim 1, wherein these syringe assembly includes a cone shaped tip to cooperate with the seal member.

4) A syringe assembly as in claim 3, wherein the cone shaped tip cooperates with the depression.

5) A syringe assembly as in claim 4, wherein the depression is a distal depression.
